(19) United States
(12) Patent Application Publication
(10) Pub. No.: US 2024/0050714 A1
(43) Pub. Date: Feb. 15, 2024

(54) Combination Balloon Catheter

(71) Applicant: Nanomedx, Inc., Santa Clara, CA (US)

(72) Inventor: Ashish Mitra, Sydney, New South Wales (US)

(21) Appl. No.: 18/260,467

(22) PCT Filed: Jan. 9, 2022
(86) PCT No.: PCT/AU2022/050006

§ 371 Date: Jul. 5, 2023

Related U.S. Application Data
(60) Provisional application No. 63/135,730, filed on Jan. 10, 2021.

(51) Int. Cl. A61M 25/10

(52) U.S. Cl. CPC A61M 25/1011; A61M 25/104

(57) ABSTRACT

Presented is an improved multifunctional balloon catheter having multiple balloons and lumens that can be inserted into a vessel (artery or vein or microvasculature) within a specific region of a human body to perform a combination of therapeutic procedures including but not limited to widening narrowed or obstructed vessel to allow an improved blood flow therethrough, perform stent/implant delivery and placement to ensure the vessel remains open, and localized/targeted delivery of various treatment/therapeutic agents to an affected area within the vessel.

### CROSS-REFERENCE TO RELATED PATENT DOCUMENTS

This patent application claims the benefit of priority of U.S. provisional Patent Application No. 63/135,730, titled “COMBINATION BALLOON CATHETER”, filed on Jan. 10, 2021, which is hereby incorporated by reference in its entirety.

### FIELD OF INVENTION

This invention relates generally to medical devices. More particularly, the present invention relates to a multifunctional balloon catheter that can be inserted into a vessel (artery or vein) within a specific region of a human body to perform multiple combination of procedures such as, procedure performed for widening narrowed or obstructed vessels to allow an improved blood flow, procedure performed for delivery of various treatment therapeutic agents to an affected area within the vessels, procedure performed for insertion of stent within the vessels to ensure the vessels remain open once it is opened by inflating the balloons of the balloon catheter, and/or a combination thereof.

### BACKGROUND

A large number of balloon catheters, which are used for expanding a stenosis site in a lumen such as a blood vessel (for example coronary artery or peripheral artery) during Percutaneous Transluminal Angioplasty (PTA) and stenting procedures are traditionally known. While using such known catheters, usually the angioplasty balloon attached over the catheter is inflated within the stenosed vessel, at the location of an atheroma or plaque deposit, in order to shear and disrupt the wall components of the vessel and widen lumen of the vessel to improve blood flow. If needed, to ensure the widened stenosis site to remain open, a stent is inserted and fixed in the vessel following the ballooning procedure.

Example of some of prior art balloon catheters includes U.S. Pat. No. 7,740,609 that discloses a catheter assembly having a guide catheter, a balloon catheter with a stabilizing balloon and a steering balloon, and an optional guide wire. As disclosed in the patent, one of two balloons may be located within a catheter, and another balloon located outside a catheter. The balloons may optionally be arranged in a co-linear relationship, disposed about the same catheter. As disclosed in the patent, while treating an occlusion in a body, firstly the catheter system with two balloons is advanced a desired site of an occlusion, secondly a first balloon is inflated to engage an interior surface of the catheter, and then finally a second balloon to engage a surface of a body lumen.

U.S. Pat. No. 5,788,708 discloses multiple balloon stent delivery catheter and method for deploying the stent in the vessels of humans.

U.S. Pat. No. 7,658,744 discloses multiple balloon catheter provided with cutting blades.

U.S. Pat. No. 4,445,892 discloses a dual balloon catheter that makes use of optic systems within blood vessels, particularly arteries. The catheter includes two spaced and expandable balloons for occluding a segment of a blood vessel. The device also includes a first channel for flushing the occluded segment, an optic system for use in the segment, and a second channel for introducing fluid into the blood vessel distally of the device.

U.S. Pat. No. 5,318,531 discloses a balloon catheter that can be used for application of medication to a blood vessel wall, for example to a stenosis site. As disclosed in the patent, the balloon includes holes of a size to permit medication delivered through the lumen to pass outwardly through the holes. The balloon carries on an outer surface a substantially hydrophilic, tubular microporous membrane covering the holes, to break up streams of flowing medication.

US20110060276 discloses a balloon catheter useful in delivering a therapeutic agent within a body vessel.

U.S. Pat. No. 6,997,898 discloses a catheter and a method for using the catheter for site specific delivery of agents to or collecting agents from biological spaces. The catheter includes inflatable balloons which when inflated in a target segment of a biological space, create closed agent delivery pockets for the delivery or collection of agents from a defined space.

U.S. Pat. No. 8,088,103 discloses a multi-lumen, multi-balloon catheter particularly used for occluding, visualizing, irrigating, evacuating, and delivering agents to a treatment area in a vessel within the human body. The catheter as proposed in the U.S. Pat. No. 103' includes elongated catheter shaft having longitudinal axis and defining five lumens therein. At its distal end, the catheter has an atraumatic tapered distal tip. The catheter includes distal occluding balloon located proximal to the tapered distal tip along the longitudinal axis of the catheter, a space-occupying balloon located proximal to the distal occluding balloon, and a proximal occluding balloon located proximal to the space-occupying balloon. Between the distal occluding balloon and the space-occupying balloon is an aspiration segment, and between the space-occupying balloon and the proximal occluding balloon is located an agent delivery segment. Each of the aspiration segment and the agent delivery segment has at least one skive port formed therein. A proximal end adapter connected to the catheter includes occluding balloon inflation hub and delivery hub. As described in the patent 103', with only the distal and proximal occluding balloons inflated and the space-filling balloon deflated, though, a relatively large treatment volume remains within the occluded vessel lumen. This large volume is particularly undesirable when scarce or expensive agents are delivered for treatment of diseased vessel; it is potentially harmful when toxic agents are to be delivered because a greater volume of those agents is required. The greater treatment volume thus produces increased expense and risk. On the other hand, inflation of the space-occupying balloon dramatically reduces the space remaining within the occluded vessel lumen, thereby increasing the effective application of agent delivered while simultaneously reducing the amount of agent needed. The space-occupying balloon is inflated to a degree that it does not contact the vessel endothelium, thus leaving the entire region of endothelium between the inflated distal and proximal occluding balloons available for exposure to the delivered agent. From the disclosure of U.S. Pat. No. 103', it is clear that the patent discloses three balloon catheter specifically for drug delivery or carrying out procedure for delivery of various treatment agents to an affected area within the vessels.

Thus, although various types of balloon catheters have existed in the past, including some with multiple balloons, for carrying out various medical procedures, the objectives of such existed balloon catheters has been to perform a single therapeutic procedure at a given time, such as PTA or stent placement or drug delivery (using coatings or using occlusive balloon catheters). If multiple therapeutic procedures were to be carried out at a specific lesion site within the vessel, the existing balloon catheters are required to be inserted within the vessel multiple times, thereby increasing the cost of treatment and requiring a patient to undergo the procedures multiple times increasing the chances of procedural complications and associated morbidities.

There is a clinical need for an improved solution, and thus inventor herein proposes a balloon catheter that can perform a combination of therapeutic procedures in a single insertion and with potentially increased safety and efficacy.

### SUMMARY

The present invention proposes an advanced balloon catheter design and method of its use thereof that would allow performing a combination of therapeutic procedures simultaneously m a single insertion and with potentially increased clinical safety and efficacy.

The inventor herein proposes a multifunctional, improved multi-balloon, and multi-lumen balloon catheter that can be inserted into a vessel within a specific region of a human body to perform a combination of therapeutic procedures such as widening narrowed or obstructed vessel to allow an improved blood flow therethrough, perform stent or an appropriate implant delivery to ensure the vessel remain open once it has been opened by inflating the balloon of the balloon catheter, and deliver various treatment therapeutic agents to an affected area within an isolated/sealed section of the vessel.

An embodiment of the present invention discloses a multi-lumen and multifunctional balloon catheter comprising: an elongated catheter body with a tip, a proximal end, and a distal end, wherein the catheter body consists of a first port, a second port, a third port, a fourth port, and a fifth port; a first balloon with a predefined shape and size is attached over the catheter and is selectively inflated and deflated within a selected blood vessel using the first port; a second balloon and a third balloon with predefined shapes and sizes attached over the catheter at the proximal end, and the distal end respectively, wherein the second and third balloons are selectively inflated and deflated within the selected vessel using the second port, and the third port respectively. Further, the fourth and fifth ports are configured for delivery and extraction of various treatment therapeutic agents and/or biological agents to and from the vessels during or after the medical procedures.

In another embodiment of the present invention, the catheter's body embodies a stent or an appropriate implant selectively configured over the first balloon. The stent or implant is inserted, delivered and inflated within the selected vessel along with the inflation of the first balloon of the catheter, and thereafter the first balloon is deflated and the catheter carrying the first balloon is optionally taken out leaving or disposing the stent at desired location within the vessel in order to ensure the widened vessel remains open once it is opened by inflating the first balloon of the balloon catheter. However, as an essence of the proposed invention, upon delivery of the stent or implant or widening of the obstructed vessel by inflated balloon, other procedure may also be simultaneously carried out such as delivery or extraction of various treatment therapeutic agents to an affected area.

According to the embodiments, when the second and third balloons attached over the catheter at the proximal end, and the distal end thereof are inflated, they create a seal at the point of contact with the vessel. This seal may be helpful in retaining the treatment agent delivered within the sealed region of the vessel for a certain period of time. This allows isolating/limiting the treatment agent between the second and third balloons, and further, allows for the complete extraction of the excess therapeutic agent thereby maximising the effect of the agent and minimizing/preventing any loss/migration of the agent to other undesirable parts of the human body.

In accordance with another embodiment, the multifunctional balloon catheter of the present invention may further be equipped with one or more electrodes within the first balloon and attached on the catheter body. The electrodes are adapted for generation of waves or vibrations that flow towards the walls of the lesion and help in impinging the lesion section adjacent to the first balloon. The waves or vibrations, such as but not limited to ultrasonic vibrations, lead to continuous vibration or drilling effect on any calcium/tough atherosclerotic lesions including calcified deposits to open up the obstructed or narrowed selected section. The waves may include mechanical waves (like sound waves, ultrasonic waves, pressure waves) capable of propagating through air, fluid, and solids, or electromagnetic waves, such as but not limited to radio frequency waves, capable of propagating through air, fluid, solid material or vacuum

These and other features, advantages and different embodiments of the present invention will become apparent from the detailed description below, in light of the accompanying drawings.

### BRIEF DESCRIPTION OF DRAWINGS

The foregoing summary, as well as the following detailed description of preferred embodiments, is better understood when read in conjunction with the appended drawings. There are shown in the drawings example embodiments, however, the application is not limited to the specific system and method disclosed in the drawings.

FIGS. 1-2 show a combination balloon catheter of the present invention, according to an exemplary embodiment of the present invention.

FIG. 3 shows a cross sectional view of the combination balloon catheter tube with a multi-port hub connected to a 5-lumen catheter tube, according to an exemplary embodiment of the present invention.

FIG. 4 shows a cross sectional view of the combination balloon catheter tube with multi-port hub connected to a 4-lumen catheter tube, according to an exemplary embodiment of the present invention.

FIG. 5 shows insertion and placement of the combination balloon catheter into a lumen of the vessel for widening of the narrowed or obstructed vessel during a ballooning procedure.

FIG. 6 shows the combination balloon catheter of FIG. 5 with a first balloon in an inflated position.

FIG. 7 shows the combination balloon catheter of FIG. 6 with the second and third balloons in inflated positions along with the first balloon to create a seal at their respective positions.

FIG. 8 shows the combination balloon catheter of FIG. 7 with the first balloon in a deflated position after the narrowed or obstructed vessel has been opened.

FIG. 9 shows the combination balloon catheter of FIG. 8 with the treatment agent delivered and retained within a sealed region formed by inflated second and third balloons.

FIG. 10 shows the combination balloon catheter of FIG. 9 with the treatment agent extracted from the sealed region.

FIG. 11 shows the second and third balloons in deflated positions after the ballooning procedure or treatment agent extraction is completed and the combination balloon catheter is to be withdrawn following the completion of medical procedure.

FIGS. 12-13 show stent delivery within the lumen of the vessel at the location of a plaque deposit or obstruction as part of the ballooning procedure that widens the obstructed vessel using the combination balloon catheter of the present invention.

FIGS. 14-15 show alternative embodiments of the proposed combination balloon catheter of the present invention that may be useful for some other specific applications.

FIG. 16 shows another alternative embodiment of the combination balloon catheter of the present invention with two electrodes configured within the balloon attached on the catheter body.

FIG. 17 shows another alternative embodiment of the combination balloon catheter of the present invention with an electrode structure in the form of a coil around the catheter body and having a plurality of wave/vibration sources.

FIG. 18 is a side view of the combination balloon catheter of FIG. 16 showing an arc between the electrodes and wave/vibration flow that helps in impinging the selected section or lesion section adjacent to the first balloon.

### DETAILED DESCRIPTION

Some embodiments, illustrating its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments are merely exemplary.

References to “one embodiment”, “an embodiment”, “another embodiment”, “an example”, “another example”, “alternative embodiment”, “some embodiment”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

As used in this application, the terms “catheter,” “balloon catheter,” “combination balloon catheter,” or the like refer to a flexible tube inserted into the human body particularly into the vessels (arteries or veins) for dilation of plaque deposits on the side walls of the vessels to improve the blood flow, delivery of the stents once the vessels are widened, and/or delivery of various treatment therapeutic agents (such as drugs, medications, etc.) within the vessels to facilitate in effectively carrying out medical procedures, recovery process, therapies and so on. Further, the terms “lesion,”, “plaque,” and so on are synonymously used for the purpose of this application and represents any deposits on the vessel wall that lead to the narrowing of the vessel that would obstruct the normal blood flow. For the purpose of this invention, the terms “selected section,”, “a point of occurrence of the lesion,”, “a lesion section,”, “calcified lesion”, etc all denotes to a diseased region or area of the blood vessel where the blood vessel has narrowed down and passage/lumen has become narrower due to deposit of plaque on the vessel wall.

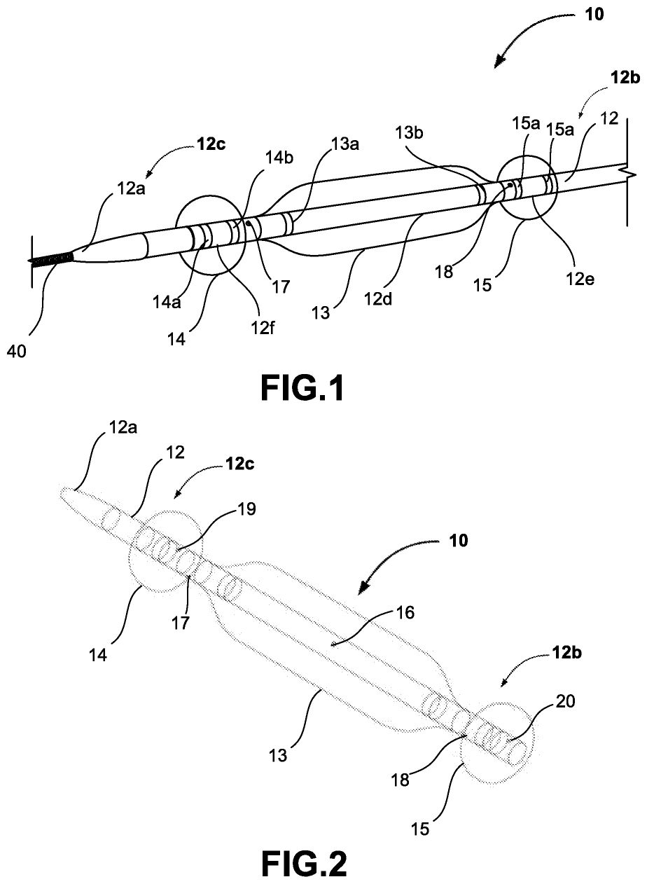

Further, the proposed catheter device that would now be described in detail with respect to FIGS. 1-15 is not limited in its use for Percutaneous Transluminal Coronary Angioplasty (PTCA), rather the proposed catheter device can be used during various Percutaneous Transluminal Angioplasty (PTA) procedures carried out to widen narrowed or obstructed arteries or veins, ranging from coronary arteries of heart to small capillaries and venules (commonly referred as “microvasculature”) and further extending to peripheral arteries and veins including the ones in the legs, arms and so on. The various features and embodiments of the present invention are better explained in conjunction with FIGS. 1-15.

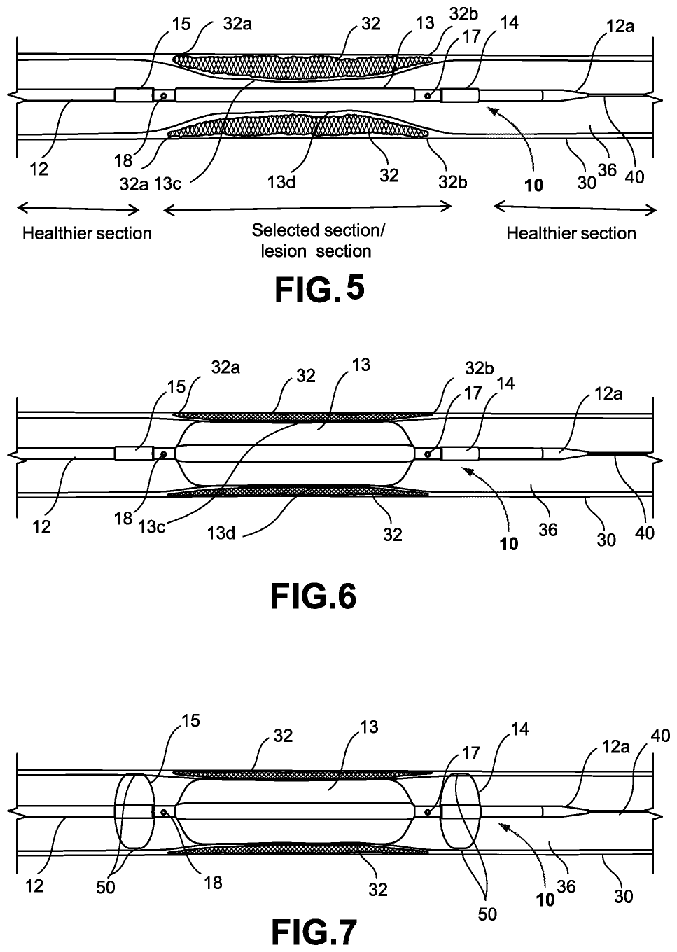

Referring to FIGS. 1-2, there is shown a multi-lumen and multifunctional balloon catheter 10. The balloon catheter 10 includes an elongated catheter body 12 having a tip 12a, a proximal end 12b, and a distal end 12c. The balloon catheter 10 includes a first balloon 13 with a predefined shape and size attached over the catheter's body 12 at a first portion 12d. The balloon 13 is selectively inflated and deflated within a selected section of a blood vessel when the medical procedure is being carried out to dilate a lesion. The balloon 13 is inflated or deflated using a first port 16 configured over the first portion 12d of the catheter body 12. The first balloon 13 when inflated for widening the selected section or lesion 32 of the blood vessel 30 pushes and comes in contact (partially or fully) with interior sides 13c, 13d of the selected section 32 of the vessel 30 (see FIGS. 5 and 6). The portion of the catheter's body 12 where the balloon 13 is attached further embodies a pair of radiopaque marker bands 13a, 13b representing either ends of the balloon 13. In other words, each of the pair of radiopaque marker bands 13a, 13b are present at two ends of the balloon 13. These radiopaque marker bands 13a, 13b helps in identifying the ends of the balloon 13 or exact position of the balloon 13 within the vessel where the medical procedure is being carried out.

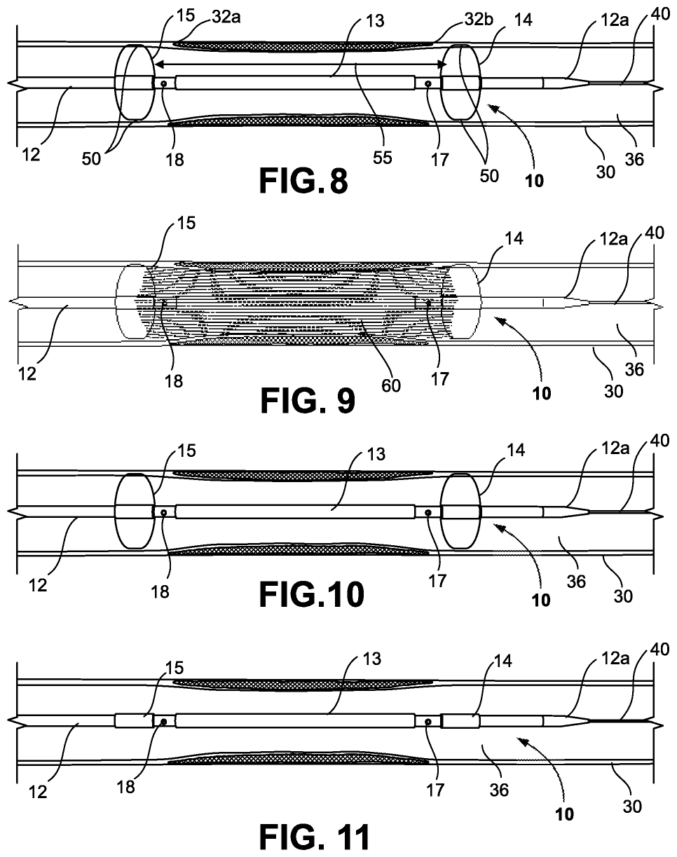

The balloon catheter 10 also includes a second balloon 15 and a third balloon 14 with predefined shapes and sizes attached over the catheter's body 12 on either sides of the first balloon 13 at the proximal end 12b, and the distal end 12c respectively. The second balloon 15 is attached on the catheter body 12 at a second portion 12e and the third balloon 14 is attached on the catheter body 12 at a third portion 12f. The balloons 14, 15 are selectively inflated and deflated within the selected section of a blood vessel where the medical procedure is being carried out to form a seal at the points of contact 50 (as shown in FIG. 8) within the blood vessel 30. The balloons 15 and 14 are inflated or deflated using a second port 20 and a third port 19 respectively. The second and third ports 20, 19 are configured over the second and third portions 12e, 12f of the catheter body 12. The second and third portions 12e, 12f of the catheter's body 12 where the balloons 15 and 14 are attached further embodies a respective pair of radiopaque marker bands 15a, 15b and 14a, 14b (referred to as a first pair of radiopaque marker bands 15a, 15b and a second pair of radiopaque marker bands 14a, 14b) configured at either ends of the balloons 15 and 14. The pairs of radiopaque marker bands 13a, and 13b, 14a, and 14b and 15a, and 15b helps the medical professionals locate ends of the balloons 13, 14, 15 respectively during the ballooning procedure. In a preferred embodiment, the balloons 14 and 15 located on either side of the balloon 13 may be identical in shape and size. In some other embodiment, the balloons 14 and 15 may differ in shapes and sizes with each other. Further, in a preferred embodiment, the size, preferably length, of the balloon 13 may be larger than the balloons 14 and 15 located on either sides of the balloon 13. In some other embodiments, the balloon 13 may be similar in size, diameter and length, to the balloons 14 and 15 located on either sides of the balloon 13.

Further, according to the embodiment, the balloon 13 may have a shape that is substantially cylindrical in nature and the balloons 14 and 15 present on either sides of the balloon 13 may have a shape that is substantially spherical in nature. Further, according to the embodiment, the balloon 13 may be made up of non-compliant or semi-compliant materials, and balloons 14 and 15 may be made of semi-compliant or compliant materials. Examples of non-compliant material include materials that can be expanded with high-pressure that are typically made of polyester or nylon and the balloon (such as the balloon 13) made of this type of material is capable of expanding to a specific diameter and exert high pressure to open a blockage or dilate the vasculature. On the other hand, examples of the compliant material, for example include but not limited to, polyurethane or silicone (elastomeric in nature). The examples of semi-compliant material include Pebax or higher-durometer polyurethanes, for example, but not limited to, ethylene-vinyl acetate, polyvinyl chloride (PVC), olefin copolymers or homopolymers, polyethylenes, polyurethanes, crosslinked low density polyethylenes (PETs), highly irradiated linear low density polyethylene (LDPE), acrylonitrile polymers and copolymers, acrylonitrile blends and ionomer resins. Other suitable balloon materials may also be used.

Further, the catheter's body 12 additionally includes a fourth port 18 and optionally a fifth port 17 located on either sides of the balloon 13 for delivery and extraction of various treatment therapeutic agents within the lesion section/selected section 32 of the blood vessel 30 during or after the medical procedures. The treatment therapeutic agents may include some specific medications, biologically active agents, anti-platelets, anticoagulants, antithrombotic and fibrolytic agents, anti-inflammatory agents, antibodies, and the like that may be delivered in a solution form individually, as a combination or in conjunction with nanoparticles such as lipid, gold, carbon and the like. Although, the preferred embodiment discloses presence of separate ports 18, 17 for delivery and extraction of treatment therapeutic agents, it may be possible to just have a common port (instead of two) for delivery and extraction of the treatment therapeutic agents. In an embodiment with separate ports 18, 17, the ports 18,17 may be connected to individual lumens (require two lumens inside the catheter body 12) to allow delivery of multiple treatment therapeutic agents, optionally simultaneously. According to the embodiment, when the balloons 14, 15 are inflated during the dilation procedure, they form the seal at the points of contact 50 (as seen in FIG. 8) with the vessel 30 to create a sealed region 55 (as seen in FIG. 8). The ports 17, 18 for injecting or delivery of the treatment agents 60 (as shown in FIG. 9) are located such that the agents 60 remain within the sealed region 55 of the vessel 30 for a certain period of time ranging from a few seconds to 15 minutes or optionally longer.

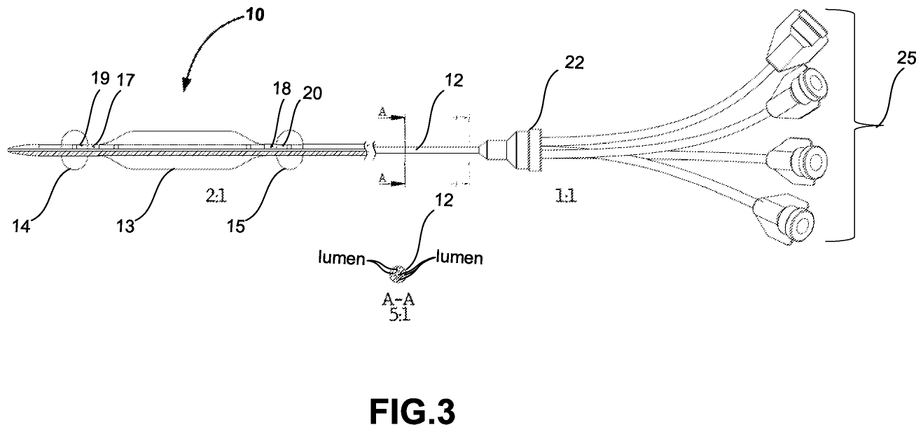

According to an embodiment, the catheter body/tube 12 may consist of 5 lumens, each lumen connected to a dedicated port 25 of the multi-port hub 22 as seen in FIG. 3. As seen in FIG. 3, the multi-port hub 22 is connected to the catheter tube 12. In the example shown, the hub 22 consists of 5 ports (although only 4 ports are visible with one guidewire lumen port unseen), each of these 4 ports may be connected to respective lumen within the catheter tube/catheter body 12 for at least inflation/deflation of the balloon 13 through the port 16, inflation/deflation of the balloon 14 through the port 19, inflation/deflation of the balloon 15 through the port 20, and delivery/extraction of treatment therapeutic agents through the ports 17, 18. Further, according to an alternative embodiment the catheter body/tube 12 may consist of 6 lumens instead of 5 lumens. Out of 6 lumens, three lumens dedicated for each of the balloons 13,14,15 for individually inflating and deflating the balloons 13,14,15, one for delivery of the treatment therapeutic agents 60, one dedicated lumen for extraction or aspiration of the treatment therapeutic agents 60 and remaining one for guidewire guidance.

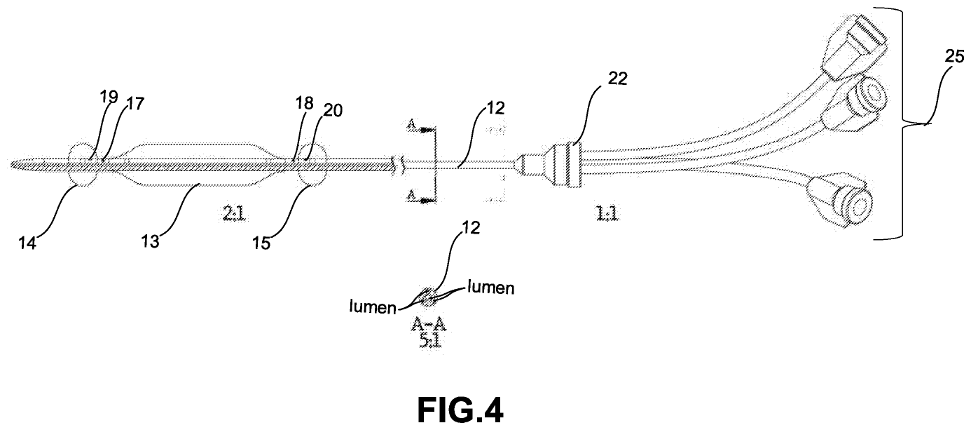

According to another embodiment, the catheter tube 12 may consist of 4 lumens instead of 5 lumen with a common lumen for inflating/deflating the balloons 14, 15, Each lumen connected to a dedicated port 25 of the multi-port hub 22. As seen in FIG. 4, the multi-port hub 22 is connected to the catheter tube 12. In the example shown, the hub 22 consists of 4 ports (although only 3 ports are visible with one guidewire lumen port unseen), each of these 3 ports of the hub 22 may be connected to respective lumen within the catheter tube 12 for at least inflation/deflation of the balloon 13 (through the port 16), inflation/deflation of the balloon 14 and 15 (through the port 19 and 20), and delivery/extraction of treatment therapeutic agents (through the ports 17, 18).

The operation of the proposed combination balloon catheter 10 and the ballooning procedure carried out using the combination balloon catheter 10 of the proposed invention will now be explained with respect to FIGS. 5-11.

In order to initiate ballooning procedure for opening the blockages or widening the narrowed or obstructed blood vessel 30 (for example, a coronary or a peripheral artery), initially a guidewire 40 is inserted into a lumen 36 of the vessel 30 to serve as a rail for the balloon catheter 10, as shown in FIG. 5. In the ballooning procedure, as known, the guidewire 40 is directed to the selected section of vessel narrowing using x-ray monitoring/visualization. Further, as seen in FIG. 5, the catheter 10 is inserted over the guidewire 40, such that the guidewire 40 passes through the central lumen of the catheter tube 12, and is placed such that the balloon 13 covers the length of the lesion or plaque 32, while the balloons 15 and 14 present on either side of the balloon 13 remain aligned with the proximal and distal edges 32a, 32b of the lesion 32 respectively. Optionally, the catheter 10 may be placed such that the balloons 15 and 14 get aligned with healthier section of the blood vessel 30 (away from the proximal and distal edges 32a, 32b of the lesion 32). Usually, based on the diameter or length of lesions the catheter 10 with appropriate balloon sizes may be chosen.

At next step, as seen in FIG. 6, the balloon 13 is inflated to open up the closed/narrowed selected section of the vessel 30 or widen the lumen 36 of the vessel 30 at the point of occurrence of lesion 32. The balloon 13 is inflated to a predetermined pressure or volume using the port 16 connected to an internal lumen of the catheter 10, that's in turn connected to a port of the hub 22 through which a suitable fluid e.g. radiopaque saline is delivered to inflate the balloon 13.

At next step, as seen in FIG. 7, following the inflation of the balloon 13, the balloons 14 and 15 located on either sides of the balloon 13 are inflated. Optionally, the balloons 14 and 15 may be simultaneously inflated. Once inflated, the balloons 14 and 15 create a seal at the points 50 of contact with the vessel 30. The balloon 14 and 15 are inflated using the ports 19, 20 connected to internal lumen of the catheter 10, that's in turn connected to ports of the hub 22 through which a suitable fluid e.g. radiopaque saline is delivered to inflate the balloons 14, 15.

At next step, as seen in FIG. 8, the balloon 13 is deflated using the same port 16. As seen, when the balloon 13 is deflated the blood vessel 30 is widened/opened at the point of occurrence of lesion 32 and remains open to allow increased blood flow. At this step, the deflation of the balloon 13 also creates a sealed region 55 between the two balloons 14 and 15.

At next step, as seen in FIG. 9, a treatment agent 60 (for example drugs, medications, nanoparticles, functionalized nanoparticles, etc.) is delivered within the sealed region 55 through the port 18. The port 18 is internally connected to a lumen of the catheter 10, the internal lumen is in turn connected to a dedicated port of the hub 22 through which the agent 60 is injected and delivered within the sealed region 55. This step may preferably be performed simultaneously with the deflation of the balloon 13 i.e. as the balloon 13 is deflated, the treatment agent 60 is delivered while continuously replacing the deflated volume of the balloon 13, to prevent any potential collapse of the vessel.

At next step, as seen in FIG. 10 (which appears same as FIG. 8), once the treatment agent 60 is delivered within the sealed region 55, the delivered agent 60 is then extracted through the port 17. In another embodiment, the agent 60 may be allowed to flow downstream (without requiring the step of extraction of the agent 60) into the blood provided the agent 60 is therapeutically viable and safe for the user/patient. At next step, as shown in FIG. 11, the balloons 14 and 15 are then deflated using the ports 19 and 20 respectively in order to unseal the sealed region 55, release and withdraw back the catheter tube 12 followed by the guide wire 40 to complete the ballooning procedure. This step may optionally be performed simultaneously with the insertion of saline i.e. as the treatment agent 60 is extracted, saline is delivered while continuously replacing the extracted volume of the treatment agent 60, to prevent any potential collapse of the vessel.

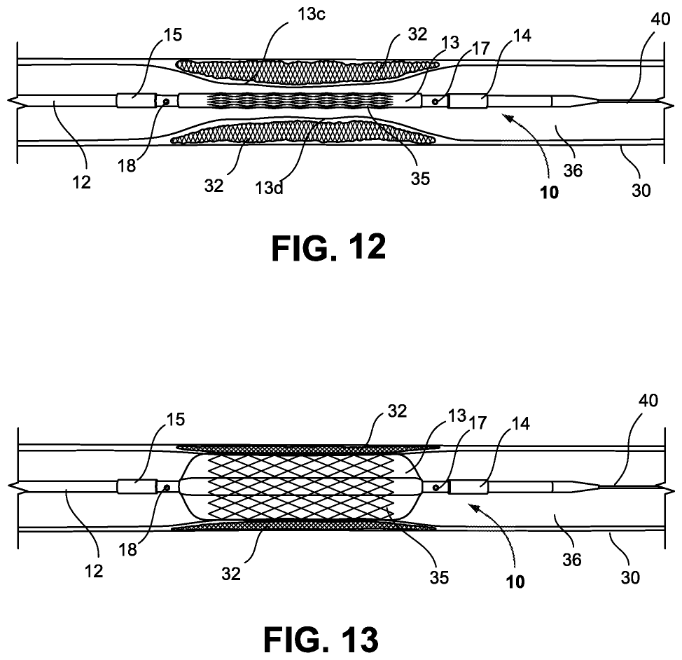

Referring to FIGS. 12-13 show the balloon catheter 10 described above with a stent 35 surrounding the balloon 13. The balloon catheter 10 of the proposed invention can be used as stent delivery system according to this embodiment. FIGS. 12-13 in particular show the stent 35 delivery within the lumen 36 of the vessel 30 at the location of the plaque/lesion deposit 32 as part of the ballooning procedure performed to widen the obstructed vessel using the combination balloon catheter 10 of the present invention.

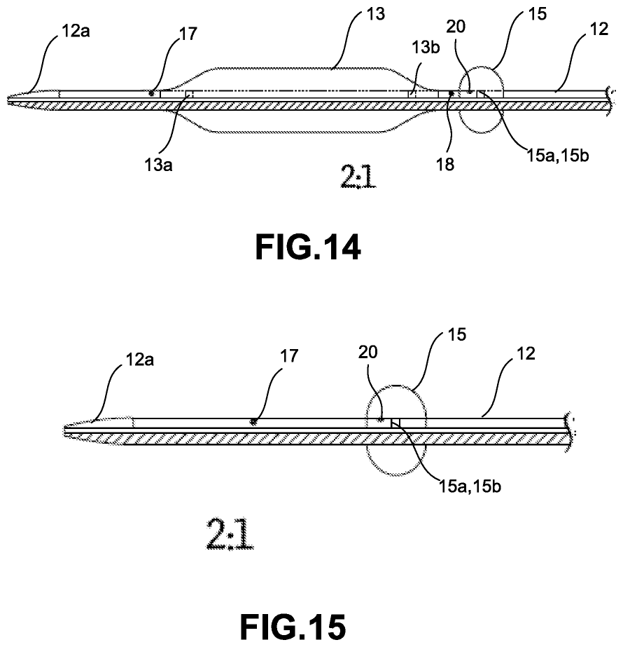

FIGS. 14-15 show alternative embodiments of the proposed combination balloon catheter 10 of the present invention that may be useful for some other specific applications. In particular, FIG. 14 shows a cross sectional view of the catheter (similar to one described above in FIGS. 1-2), with just the presence of balloon 13 and the balloon 15. The embodiment represented by FIG. 14 can optionally be used for treatment of coronary artery lesions, specifically the lesions that occur near the distal end of the arteries. Port 17 could be present on either side of the balloon 13 in this embodiment. Optionally, the balloon 15 may only maintain a partial seal to allow continued partial blood supply distally. Likewise, FIG. 15 shows a cross sectional view of the catheter (similar to one described above in FIGS. 1-2), with just the presence of the balloon 15. This embodiment of the proposed combination balloon catheter 10 is preferably deployed for the treatment of microvasculature i.e. delivery of the treatment therapeutic agents to the microvasculature e.g. the coronary microvasculature. The balloon 15 when inflated for widening the selected section or lesion 32 of the blood vessel 30 and delivery of the one or more treatment therapeutic agents 60 within the selected section 32 of the blood vessel 30 pushes and comes in contact (partially or fully) with interior sides 13c, 13d of the selected section or selected lesion 32 of the vessel 30. In these embodiments, the balloon 15 may not be used for widening the lesion section 32.

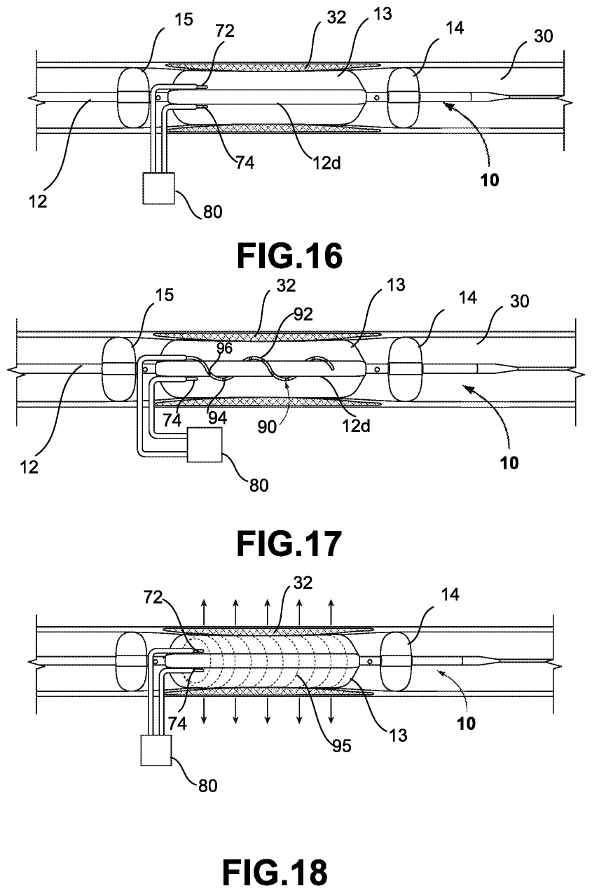

FIGS. 16 and 17 show alternative embodiments of the present invention. In particular, FIG. 16 shows the combination balloon catheter 10 of the present invention with two electrodes configured within the first balloon and attached on the catheter body. As seen, the catheter 10 is similar to the one explained above with respect to FIGS. 1-13 except the presence of electrodes 72 and 74. The electrodes are attached to the catheter body 12 (located on the first portion 12d). The electrodes 72, 74 are further attached to a source of high voltage pulses 80. The electrodes 72 and 74 are formed of metal and are placed a distance apart to allow a reproducible arc for a given voltage and current. The electrical arcs between electrodes 72 and 74 in the fluid (that helps in inflating the balloon 13) are used to generate waves or vibrations in the fluid. These pulses/waves lead to continuous vibration or drilling effect on any calcium/tough atherosclerotic lesions including calcified deposits to open up the obstructed or narrowed selected section. The waves may include mechanical waves (like sound waves, ultrasonic waves, pressure waves) capable of propagating over air, water/fluid, and metals, or electromagnetic waves capable of propagating over air, solid material or vacuum. The variable power source 80 is used to deliver a stream of pulses to the electrodes 72 and 74 to create a stream of waves or vibrations within the balloon 13 and within the selected section 32 of the vessel 30 with lesion being treated. The magnitude of the waves can be controlled by controlling the magnitude of the pulsed voltage, the current, and the duration and repetition rate. FIG. 18 shows a side view of the combination balloon catheter of FIG. 16 showing an arc between the electrodes and wave flow. The waves or vibrations help strike the selected section or lesion section adjacent to the first balloon 13. The generated waves or vibrations 95 propagate through the fluid and continuously strike the lesion section. This action helps in widening of the narrowed or obstructed selected section 32 with ease. In many cases, especially in the patients with old history of narrowed or calcified lesion 32, mere inflation of the balloon 13 may not be sufficient to widen up the calcified lesion 32 of the vessel 30. Thus, in such cases application of the waves 95 together with the inflation of the balloon 13 will generate more pressure on the interior side walls of the lesion 32 thereby increasing chances of widening narrowed or obstructed vessels to allow an improved blood flow therethrough.

FIG. 17 shows another alternative embodiment of the combination balloon catheter of the present invention with an electrode structure configured in the form of a coil around the catheter body and having a plurality of wave sources. Like the embodiment shown in FIG. 16, the catheter 10 includes an electrode structure 90 in the form of a coil. The electrode structure 90 configured on the first portion 12d of the catheter body 12. The electrode structure 90 includes an insulated wire 92 wound about the catheter body 12. The insulated wire 92 may include a plurality of openings 94 that expose corresponding discrete portions of the insulated wire conductor to the fluid (e.g. saline) filled within the balloon 13 for inflation. Each opening 94 forms corresponding wave source/electrodes 96. The electrode structure 90 also includes a counter electrode 74. The counter electrode 74 serves as a common electrode to cause an electrical arc to occur between each of the electrodes 96 and the electrode 74 when a suitable high voltage is applied between the electrodes 96 and the counter electrode 74. As explained above, the electrodes 74 and 96 are attached to a source 80 of high voltage pulses. The variable high voltage pulse generator 80 is used to deliver a stream of pulses to the electrodes 96 and 74 to create a stream of waves or vibrations within the balloon 13 and within the selected section 32 of the vessel 30 with lesion being treated.

The combination balloon catheter 10 and associated parts thereof such as the balloons 13,14,15, and the other facilitating means such as multiport hub 22 connected to the catheter's tube may be made using wide range of materials in different dimensions.

The preceding description has been presented with reference to various embodiments. Persons skilled in the art and technology to which this application pertains will appreciate that alterations and changes in the described structures and methods/steps of operation can be practiced without meaningfully departing from the principle, spirit and scope of the present invention.

1. A multifunctional balloon catheter, comprising:
an elongated catheter body having a tip, a proximal end, and a distal end;
a first balloon with a predefined shape and size attached over a first portion of the elongated catheter body;
a second balloon, and a third balloon with predefined shapes and sizes attached over a second portion and a third portion of the catheter body on either side of the first balloon at the proximal end, and the distal end of the catheter body respectively;
wherein, the first balloon, the second balloon, the third balloon are selectively inflated or deflated to perform at least one of: a procedure for widening a selected section or a point of occurrence of lesion of a blood vessel in order to allow an improved blood flow therethrough, a procedure for delivery of one or more treatment therapeutic agents within the selected section of the blood vessel, and a procedure for deployment of a stent or implant within the selected section of the vessel;
wherein, the second balloon and the third balloon when inflated within the selected section of the blood vessel form a sealed region extending in between the points of contact of the second balloon and the third balloons with the blood vessel; and
wherein, the first balloon when inflated for widening the selected section or lesion of the blood vessel pushes and comes in at least partial contact with interior sides of the selected section of the vessel.

2. The multifunctional balloon catheter of claim 1, wherein the elongated catheter body includes a first port located on the first portion for selectively inflating or deflating the first balloon within the selected section of the blood vessel when the medical procedure is carried out to dilate a lesion.

3. The multifunctional balloon catheter of claim 1, wherein the first portion of the elongated catheter body having the first balloon attached thereon comprises a pair of radiopaque marker bands.

4. The multifunctional balloon catheter of claim 1, wherein the second balloon, and the third balloon are selectively inflated or deflated using a second port and a third port configured over the second portion and the third portion of the catheter body respectively.

5. The multifunctional balloon catheter of claim 4, wherein the inflation of the second balloon and the third balloon form a seal at their points of contact with the selected section of the blood vessel.

6. The multifunctional balloon catheter of claim 4, wherein each of the second portion and the third portion of the catheter body having the second balloon and the third balloon attached thereon further comprises a first pair of radiopaque marker bands and a second pair of radiopaque marker bands respectively configured at either ends of the second balloon and third balloon.

7-11. (canceled)

12. The multifunctional balloon catheter of claim 1, wherein the first balloon is made up of non-compliant or semi-compliant materials capable of expanding to a specific diameter and exerting a high pressure onto the walls of the blood vessel, wherein the non-compliant or semi-compliant materials are selected from the group consisting of polyester, nylon, polyurethane, and silicon.

13. (canceled)

14. The multifunctional balloon catheter of claim 1, wherein the second balloon and the third balloon are made up of semi-compliant or compliant materials wherein the semi-compliant or compliant materials are selected from the group consisting of polyurethane, silicon, ethylene-vinyl acetate, polyvinyl chloride, olefin copolymers or homopolymers, polyethylenes, polyurethanes, crosslinked low density polyethylenes, highly irradiated linear low density polyethylene, acrylonitrile polymers and copolymers, acrylonitrile blends, and ionomer resins.

15. (canceled)

16. The multifunctional balloon catheter of claim 1 further comprising at least one port located either on one side or both the sides of the first balloon for delivery and extraction of one or more treatment therapeutic agents within the selected section of the blood vessel during or after the medical procedures.

17. The multifunctional balloon catheter of claim 16, wherein the one or more treatment therapeutic agents comprises medications, biologically active agents, anti-platelets, anticoagulants, antithrombotic and fibrolytic agents, anti-inflammatory agents, antibodies deliverable in a solution form either individually, or as a combination or in conjunction with nanoparticles including lipid, gold, carbon.

18. The multifunctional balloon catheter of claim 1, wherein the first balloon is further configured to carry the stent deployable at the selected section or the point of occurrence of lesion by inflating the first balloon.

19. (canceled)

20. The multifunctional balloon catheter of claim 1, wherein the first balloon is sized to substantially cover the length of the selected section or lesion of the blood vessel while the second balloon and the third balloon remain aligned within or at a proximal edge and within or at a distal edge of the selected section or lesion of the blood vessel.

21. The multifunctional balloon catheter of claim 1, wherein the first balloon is sized to substantially cover the length of the selected section or lesion of the blood vessel while the second balloon and the third balloon remain aligned away from the proximal edge and a distal edge of the selected section or lesion of the blood vessel towards healthier sections of the blood vessel.

22. The multifunctional balloon catheter of claim 1 further comprising one or more electrodes attached or located in close proximity to the first portion of the catheter body and powered by a power source, wherein the power source delivers a stream of pulses to the one or more electrodes to create a stream of waves or vibrations within the first balloon that migrate towards the selected section or lesion section of the blood vessel.

23. (canceled)

24. The multifunctional balloon catheter of claim 22, wherein the stream of waves or pulses lead to continuous vibration or drilling effect on the lesion section of the blood vessel.

25. The multifunctional balloon catheter of claim 22, wherein the waves comprising at least mechanical waves including sound waves or ultrasonic waves or pressure waves capable of propagating through air, fluid, and solids; or electromagnetic waves capable of propagating through air, fluid, solid material or vacuum.

26. A multifunctional balloon catheter used for treatment of lesions that occur near the distal end of the arteries or veins, comprising:
an elongated catheter body having a tip, a proximal end, and a distal end;
a first balloon with a predefined shape and size attached over a first portion of the elongated catheter body;
a second balloon with a predefined shape and size attached over a second portion of the catheter body at the proximal end thereof;
wherein, the first balloon, the second balloon are selectively inflated or deflated to perform at least one of: a procedure for widening a selected section or a point of occurrence of lesion of a blood vessel in order to allow an improved blood flow therethrough, a procedure for delivery of one or more treatment therapeutic agents within the selected section of the blood vessel, and a procedure for deployment of a stent within the selected section or lesion section of the vessel to ensure the selected section of the blood vessel remain open once it is opened by inflating the first balloon of the balloon catheter;
wherein, the second balloon when inflated within the selected section of the blood vessel form a partial seal to allow continued partial blood supply distally at the end of the blood vessel; and
wherein, the first balloon when inflated for widening the selected section or lesion of the blood vessel pushes and comes in at least partial contact with interior sides of the selected section or selected lesion of the vessel.

27-30. (canceled)

31. A multifunctional balloon catheter used for treatment of microvasculature, comprising:
an elongated catheter body having a tip, a proximal end, and a distal end;
a balloon with a predefined shape and size attached over a portion of the catheter body at an end;
wherein, the balloon is selectively inflated or deflated to perform at least one of: a procedure for widening a selected section or a point of occurrence of lesion of a blood vessel in order to allow an improved blood flow therethrough, a procedure for delivery of one or more treatment therapeutic agents within the selected section of the blood vessel, and a procedure for deployment of a stent within the selected section or lesion section of the vessel to ensure the selected section of the blood vessel remain open once it is opened by inflating the first balloon of the balloon catheter; and
wherein, the balloon when inflated for widening the selected section or lesion of the blood vessel and delivery of the one or more treatment therapeutic agents within the selected section of the blood vessel pushes and comes in at least partial contact with interior sides of the selected section or selected lesion of the vessel.

32. The multifunctional balloon catheter of claim 31 further comprising one or more electrodes attached or located in close proximity to the first portion of the catheter body and powered by a power source.

33-35. (canceled)

36. The multifunctional balloon catheter of claim 1, wherein the catheter is configure to inflate the first balloon to widen a selected section or a point of occurrence of lesion of a blood vessel, prior to inflating the second balloon and the third balloon on either side of the first balloon to create a sealed region between their points of contact with the blood vessel, and then deflate the first balloon before delivering one or more treatment therapeutic agents (60) to the sealed region through at least one port located at one or both sides of the first balloon.
